(19) United States
(12) Patent Application Publication
(10) Pub. No.: US 2025/0032778 A1
(43) Pub. Date: Jan. 30, 2025

(54) MEDICAL LEAD SPLINE

(71) Applicant: Medtronic, Inc., Minneapolis, MN (US)

(72) Inventors: Damian M. Becker, Columbia Heights, MN (US); Sajith Anantharaman, Maple Grove, MN (US); Jeffrey M. Novotny, North Branch, MN (US)

(21) Appl. No.: 18/360,949

(22) Filed: Jul. 28, 2023

(51) Int. Cl. A61N 1/05

(52) U.S. Cl. CPC A61N 1/05

(57) ABSTRACT

A lead assembly comprising: a plurality of conductors; a spline comprising: a plurality of projections disposed around an outer perimeter of a spline body and extending radially away from an outer surface of the spline body, wherein each projection of the one or more projections comprises two or more projection segments separated by one or more longitudinal gaps, wherein the plurality of projections define a plurality of conductor channels around the outer perimeter of the spline body, wherein each conductor of the plurality of conductors is disposed within a respective conductor channel of the plurality of conductor channels; a plurality of electrical contacts electrically coupled to respective conductors of the plurality of conductors at the conductor first end; and an outer lead body disposed radially outwards from at least a portion of the spline body and at least a portion of each conductor of the plurality of conductors.

### TECHNICAL FIELD

The present disclosure relates to medical devices, more particularly to medical leads configured for delivering electrical signals and/or sensing electrical signals.

### BACKGROUND

Implantable electrical stimulators have been proposed for use to treat a variety of symptoms or conditions, such as chronic pain, tremor, Parkinson's disease, epilepsy, urinary or fecal incontinence, sexual dysfunction, obesity, or gastroparesis. An electrical stimulator may be configured to deliver electrical stimulation therapy to a patient via one or more medical leads that include electrodes implanted proximate to a target tissue within the patient, such as a target tissue site proximate the spinal cord, pelvic nerves, peripheral nerves, or within the brain or stomach of a patient. Hence, electrical stimulation may be used in different therapeutic applications, such as deep brain stimulation (DBS), spinal cord stimulation (SCS), pelvic stimulation, gastric stimulation, or peripheral nerve stimulation. Stimulation also may be used for muscle stimulation, e.g., functional electrical stimulation (FES) to promote muscle movement or prevent atrophy.

Electrical stimulation may be delivered via one or more implanted or percutaneous leads, each lead carrying one or more electrodes. The electrodes may take the form of, e.g., ring electrodes, cuff electrodes, paddle electrodes, segmented ring electrodes. Leads may be constructed, for example, by welding each electrode to a conductor (e.g., a wire) disposed within a lead body of the lead. When completed, an electrical signal generated by the electrical stimulator may be transmitted through one or more conductors and respective electrodes of the lead to generate an electrical field within the patient.

### SUMMARY

In general, the disclosure is directed to devices, systems, and techniques for fabricating a medical lead, which may include multiple electrodes. The devices, systems, and techniques allow for conductor separation for election isolation via protrusions along a spline body extending along the medical lead. The electrical isolation of electrical conductors assists in improving impedance values for the lead. The spline body may define one or more different configurations as described in this disclosure to facilitate manufacture or assembly of the medical lead. The spline body configurations may include, but are not limited to, spline bodies with separate protrusions segments separated by longitudinal gaps, spline bodies with protrusions defining conductor channels defining a helical pattern, or spline bodies with protrusions of varying heights. The spline body configurations described herein may reduce a likelihood of deformation of the spline body during manufacture, facilitate placement of electrodes over the spline body during medical lead assembly, and/or reduce the number of components and/or steps required to manufacture the medical lead.

In one example, this disclosure describes a lead assembly comprising: a plurality of conductors, each conductor of the plurality of conductors extending from a conductor first end to a conductor second end along a longitudinal axis; a spline extending from a spline first end to a spline second end, the spline comprising: a spline body extending along the longitudinal axis, and a plurality of projections disposed around an outer perimeter of the spline body, the plurality of projections extending radially away from an outer surface of the spline body, wherein one or more projections of the plurality of projections extend along the longitudinal axis, and wherein each projection of the one or more projections comprises two or more projection segments separated by one or more longitudinal gaps, wherein the plurality of projections define a plurality of conductor channels around the outer perimeter of the spline body, wherein each conductor of the plurality of conductors is disposed within a respective conductor channel of the plurality of conductor channels; a plurality of electrical contacts electrically coupled to respective conductors of the plurality of conductors at the conductor first end; and an outer lead body disposed radially outwards from at least a portion of the spline body and at least a portion of each conductor of the plurality of conductors.

In another example, this disclosure describes a method of constructing a lead assembly, the method comprising: disposing a plurality of conductors within a plurality of conductor channels in a spline, the spline comprising: a spline body extending along a longitudinal axis, and a plurality of projections disposed around an outer perimeter of the spline body, each projection extending radially away from an outer surface of the spline body, wherein one or more projections of the plurality of projections extend along the longitudinal axis, and wherein each projection of the one or more projections comprises two or more projection segments separated by one or more longitudinal gaps, wherein the plurality of projections define the plurality of conductor channels along at least a portion of the longitudinal length of the spline body; disposing a plurality of electrical contacts over the spline body; electrically coupling each conductor of the plurality of conductors to a respective electrical contact of the plurality of electrical contacts; and disposing an outer lead body radially outwards from at least a portion of the spline body and at least a portion of each conductor of the plurality of conductors.

In another example, this disclosure describes a method of constructing a spline, the method comprising: forming a spline body extending along a longitudinal axis; forming a plurality of projections extending from an outer surface of the spline body and around an outer perimeter of the spline body; and ablating portions of one or more projections of the plurality of projections until each of the one or more projections defines two or more projection segments separated by one or more longitudinal gaps, wherein the plurality of projections define a plurality of conductor channels along the outer perimeter of the spline body, wherein each conductor channel of the plurality of conductor channels is configured to retain a respective conductor of a plurality of conductors.

The details of one or more examples are set forth in the accompanying drawings and the description below. Other features, objects, and advantages will be apparent from the description, drawings, and claims.

### BRIEF DESCRIPTION OF DRAWINGS

The details of the present disclosure are set forth in the accompanying drawings and the description below. Other features, objects, and benefits will be apparent from the description and drawings, and from the claims.

FIG. 1A is a conceptual diagram illustrating an example medical lead with ring electrodes.

FIG. 1B is a conceptual diagram illustrating an example medical lead with three segmented ring electrodes in the middle two electrode levels.

FIG. 1C is a cross-section of the lead of FIG. 1A at the distal end of the lead along line A-A in FIG. 1A.

FIG. 1D is a cross-section of the lead of FIG. 1A at the distal end of the lead along line A-A in FIG. 1A.

FIG. 1E is a conceptual diagram of a electrodes disposed along the distal end of the lead.

FIG. 2A is a partial cross-section of an example of the lead of FIG. 1A at a first end of the lead.

FIG. 2B is a partial cross-section of the example lead of FIG. 1A along a medial portion of the lead.

FIG. 3A is a partial cross-section of another example of the lead of FIG. 1A at the distal end of the lead.

FIG. 3B is a partial cross-section of the example lead of FIG. 1A along a medial portion of the lead.

FIG. 4A is a partial cross-section of another example of the lead of FIG. 1A at the distal end of the lead.

FIG. 4B is a partial cross-section of the example lead of FIG. 1A along a medial portion of the lead.

FIG. 5A is a partial cross-section of another example of the lead of FIG. 1A at the distal end of the lead.

FIG. 5B is a partial cross-section of the example lead of FIG. 1A along a medial portion of the lead.

FIG. 6A is a cross-section of the lead of FIG. 2A along line B-B in FIG. 2A.

FIG. 6B is a cross-section of the lead of FIG. 2B along line C-C in FIG. 2B.

FIG. 7A is a cross-section of the lead of FIG. 3A along line D-D in FIG. 3A.

FIG. 7B is a cross-section of the lead of FIG. 4A along line E-E in FIG. 4A.

FIG. 8 is another example cross-section of the lead of FIG. 2A along line B-B in FIG. 2A.

FIG. 9 is another example cross-section of the lead of FIG. 2A along line B-B in FIG. 2A.

FIG. 10 is another example cross-section of the lead of FIG. 2A along line B-B in FIG. 2A.

FIG. 11 is a flow chart illustrating an example process of manufacturing a medical lead, in accordance with the examples described in this disclosure.

### DETAILED DESCRIPTION

Devices, systems, and techniques for fabricating a medical lead with one or more electrodes are described herein. In some examples of electrical stimulation therapy, a therapy system includes a medical device configured to generate electrical stimulation signals and a medical lead to deliver or transfer the stimulation signals to the patient. The lead may include one or more electrodes (e.g., disposed on a longitudinal surface, distal tip, or both of the lead) configured to deliver the electrical stimulation signals to the patient.

As disclosed herein, various structures and techniques may be utilized to electrically isolate the conductors within the lead, which may improve impedance values. As leads become more sophisticated, impedance requirements have increased, as well as the need to better isolate conductors, connectors, and electrodes. Increasing the isolation of the conductors, connectors, and electrodes may improve impedance characteristics, increase control over electrode alignment, facilitate lead assembly with higher densities of electrodes, and decrease lead variability (e.g., variability between leads of the same type).

Electrical separation of the wires, or conductors, can be done via insulation on the wires and/or other separation devices at certain locations along the lead. A spline may be located at the distal end of the lead near the electrodes. In addition, heat shrink tubing may be provided over the transition of the proximal end of the spline and middle portion of the lead to relieve stress. Such heat shrink tubing may also enable injection molding or overmolding of both ends of the lead at the same time because the heat shrink tubing retains the positions of the conductors.

The leads described herein may be used to deliver a variety of electrical stimulation therapies to a patient. In one example, the lead may be used to deliver neurostimulation therapy to a patient's brain, e.g., DBS. However, the features and techniques described herein are useful in other types of medical device systems, which may include other types of implantable medical leads and implantable medical devices. For example, the fabrication devices and techniques described herein may be used to fabricate cardiac leads for cardiac rhythm management devices (e.g., pacemakers or pacemaker-cardioverter-defibrillators). As other examples, the features and techniques described herein may be used for leads that deliver other types of neurostimulation therapy (e.g., spinal cord stimulation or vagal stimulation), stimulation of at least one muscle or muscle groups, stimulation of at least one organ such as gastric system stimulation, stimulation concomitant to gene therapy, and, in general, stimulation of any tissue of a patient.

In addition, leads described herein may be coupled at their proximal ends to a stimulation therapy controller (e.g., an implantable medical device) located remotely from the electrodes, but other configurations are also possible and contemplated. For example, a lead may be defined, at least in part, by a portion of a housing (e.g., a medical device housing) or a member coupled to a housing of a medical device. In another example, the lead may even include a stimulation generator, e.g., a microstimulator, located proximate to or at the stimulation site. In other examples, a lead may include a member at a stimulation site that is wirelessly coupled to an implanted or external stimulation controller or generator. The processes, devices, and systems described herein for fabricating a medical lead may be used with any medical device that includes electrodes may disposed on a surface of the device.

As described herein, axial, radial, and circumferential directions refer to a cylindrical coordinate system with respect to the lead that is being fabricated. In other words, the axial direction is the longitudinal direction parallel with a center axis defined by the lead. The radial direction is the direction orthogonal to or at a right angle to the center axis. In other words, the radial direction extends directly away from the center axis. The circumferential direction refers to the angular position or direction around the outer surface of the lead. Different circumferential positions with respect to the lead may vary by some angle centered at the center axis.

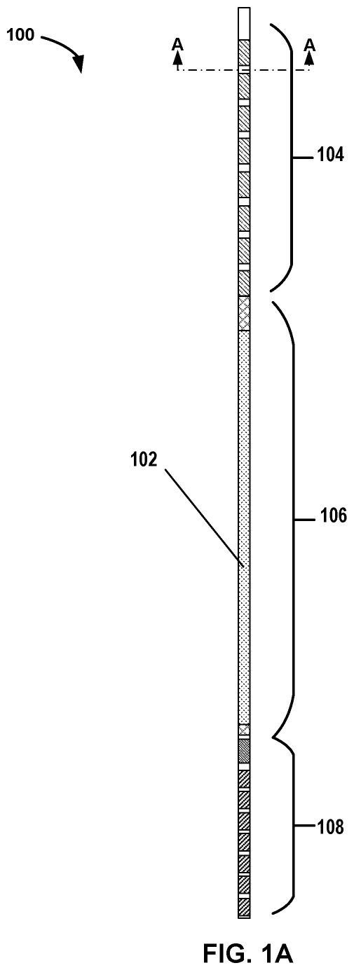

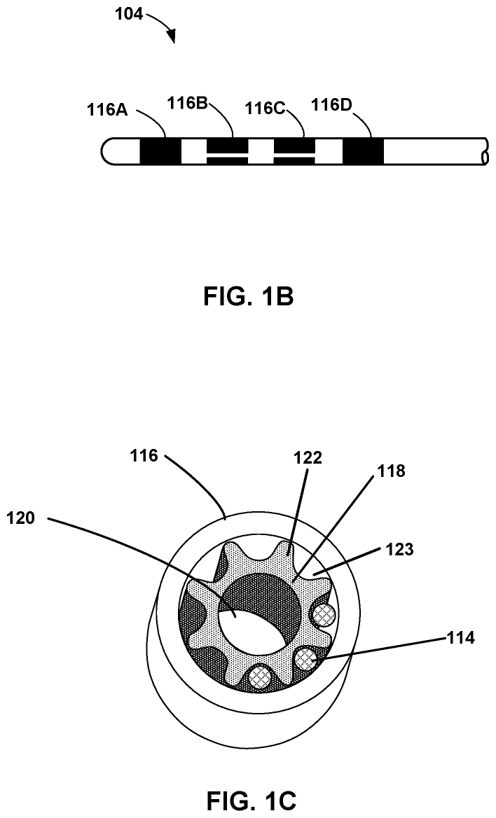

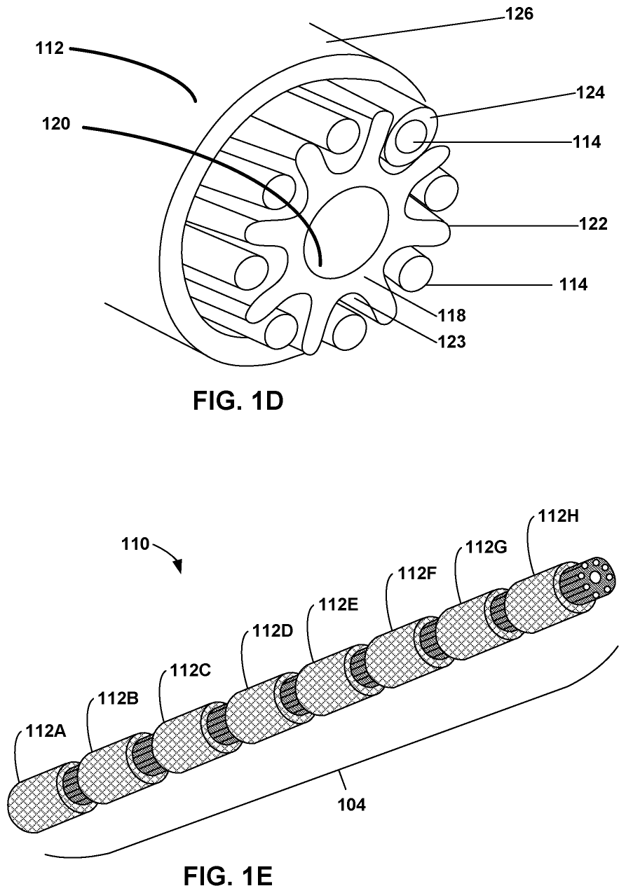

FIGS. 1A-1E illustrate example leads that may be fabricated in accordance with the processes, devices, and systems described herein. FIG. 1A is a conceptual diagram illustrating an example medical lead 100, that includes a lead body 102 including a first lead end 104, a lead medial portion 106, and a second lead end 108, where the lead medial portion 106 is between the first lead end 104 and the second lead end 108. In some examples, the first lead end 104 is a distal lead end configured to be disposed in contact with tissue of a patient. In one or more examples, the second lead end 108 is a proximal lead end configured to be coupled with an implantable medical device. Lead 100 may include contacts, such as ring electrodes or segmented ring electrodes (e.g., electrodes disposed only partially around the perimeter or circumference of the lead). As shown in FIG. 1A and FIG. 1E, lead 100 may include outer lead body 102 and a first set of contacts 112 which may include contacts 112A, 112B, 112C, 112D, 112E, 112F, 112G, and 112H (collectively referred to as “contacts 112”). In some examples, the first set of contacts 112 are disposed at a first, for example distal, lead end 104, and may be configured to contact tissue. The lead 100 may further include a second set of contacts disposed at second lead end 108. The second set of contacts may be configured to be connected to an implantable medical device. In one or more examples, the second set of contacts are electrically coupled with the implantable medical device.

In one or more examples, the first set of contacts 112 may comprise electrodes such as ring electrodes at the different electrode levels. FIG. 1B illustrates another example of the first lead end 104 which may include segmented electrodes 116 for at least some of the first set of contacts 112. In the example shown in FIG. 1B, electrode levels 116A and 116D each includes a single ring electrode within the respective level. Electrode levels 116B and 116C each includes three segmented ring electrodes disposed at different positions around a perimeter of the lead. Each of electrodes 116 may have a separate and distinct conductor within lead 104 to a respective contact at the opposite end of the lead. Lead 100 having electrode levels as shown in FIG. 1B may be referred to as a “1-3-3-1” lead because each electrode level comprises one, three, three, and one electrode, respectively.

Contacts 112 may define an outer surface and an inner surface. The outer surface may be configured to contact tissue of a patient. The inner surface may define a contact lumen configured to receive one or more lead components (e.g., conductors, splines, stylets). Lead 100 may also be described as including a complex electrode array geometry. A complex electrode array geometry may be an electrode array that includes at least one level of segmented ring electrodes (e.g., circumferentially positioned electrodes). In another example, a complex electrode array geometry may refer to an electrode array that includes electrodes centered in two, three, or even more planes. A complex electrode geometry may indicate any electrode array in which different electrode combinations may be used to deliver electrical stimulation in multiple directions away from the lead. Thus, the complex electrode array geometry may include multiple levels of segmented ring electrodes, segmented ring electrodes and ring electrodes, or any other combination of electrodes including at least one level of segmented ring electrodes. Segmented ring electrodes may generally be two or more electrodes located at different angular or circumferential positions around the circumference of lead body 102. Segmented ring electrodes or other complex electrode array geometries may be used to produce customizable stimulation fields (e.g., electrical fields that may affect or activate patient tissue) that may be directed to a particular side of lead 100 in order to isolate the stimulation field around the target anatomical region of a brain in DBS, for example.

Lead 100 may have any suitable configuration. Lead 100 may be substantially cylindrical (e.g., cylindrical or nearly cylindrical) in shape (e.g., may have a circular or nearly circular cross-section when the cross-section is taken in a direction perpendicular to a longitudinal axis of lead 100). In other examples, however, lead 100 may have another suitable shape. For example, lead 100 may define one or more curves, e.g., a shape configured to reach target anatomical regions of the patient. In some examples, lead 100 may be similar to a flat paddle lead or a conformable lead shaped for the patient. Also, in other examples, lead 100 may for constructed of any of a variety of different polygonal cross sections taken transverse to the longitudinal axis of the lead. Although lead 100 may be generally flexible, a lead may include one or more portions that are semi-rigid or rigid to aid in implantation and/or achieve desired orientation of lead 100 within the patient.

Outer lead body 116 may be formed from an insulative biocompatible material. Example biocompatible materials may include at least one of polyurethane, silicone, and fluoropolymers such as tetrafluoroethylene (TFE), polytetrafluoroethylene (PTFE), and/or expanded PTFE (i.e., porous ePTFE, nonporous ePTFE). Outer lead body 116 may be a molded lead body that at least partially surrounds a lead structure that supports the electrodes and plurality of conductors (e.g., electrically conductive wires) that electrically couple to respective electrodes of lead 100. In some examples, outer lead body 116 may be injection molded or overmolded.

Within outer lead body 116, lead 100 may also include a plurality of conductors 114. In some examples, the plurality of conductors 114 may each include a respective insulation sheath. In some examples, the conductors may have SI polyamide insulation. In some examples, the insulation may enable direct welding between an electrode and a conductor without separate ablation of the insulation. In some examples, the welding process may burn through the insulation as it combines the metals together. Conductors 114 may extend from a conductor first end to a conductor second end. In some examples, the outer lead body 116 is disposed over (e.g., be dispose radially outwards from) at least an intermediate portion of conductors 114. In some examples, the conductors 114 terminate at the respective contacts. For example, the conductor first end of conductors 114 may in contact with corresponding contacts 112 of the first set of contacts and/or the conductor second end of conductors 114 may be in contact with corresponding contacts of the second set of contacts. In some examples, conductors 114 may include a slip tube 124, as shown in FIG. 1D, and the conductor may be laser welded at 126 to electrically connect conductors 114 with an inner surface of the contacts 112.

In some examples, the conductors may be coiled along part or all of the length of lead body 102 (e.g., in a multiconductor coil). In some examples, the conductors may be substantially straight instead of coiled. In some examples, each of conductors 114 may define a helix, spiral, or coil around the outer perimeter of lead body 102 and along the longitudinal length of lead body 102. In such examples, each of conductors 114 may define less than one, one, or more than one complete revolutions around the outer perimeter of lead body 102. The conductors may be straightened or curved at their distal end (e.g., near their respective electrode) to be mechanically coupled to an electrode.

As shown in FIG. 1C, lead 100 includes a spline 118 within lead body 102. Spline 118 may be disposed within first lead end 104 of lead 100. Spline 118 includes a spline body extending from a first spline end to a second spline end. Spline 118 may include a plurality of protrusions 122 extending from an outer surface of the spline and defining a plurality of channels 123 between adjacent protrusions. Each of channels 123 may be configured to retain a respective conductor of the plurality of conductors 114. Conductor channels 123 are sized and configured to receive conductors 114 therein. Protrusions 122 may electrically isolate circumferentially adjacent conductors 114. In some examples, conductor channels 123 extend from a first end of spline 118 to a second end of spline 118. In some examples, the conductor first end of each conductor of the plurality of conductors 114 is disposed within respective conductor channels of the conductor channels 123.

In some examples, the spline 118 may be a straight spline (e.g., protrusions 122 extend along a longitudinal axis of spline 118), or may be twisted spline (e.g., protrusions 122 define a helix or spiral around the longitudinal axis of spline 118). In one or more examples, spline 118 may be a nine-lumen spline which may have eight conductor channels 123 and a stylet lumen 120, as shown in FIGS. 1C and 1D. Fewer or greater numbers of protrusions may be used in other examples. In one or more examples, the spline 120 may be disposed at the lead distal end. In one or more examples, the spline 120 may be disposed along the lead body 102, for example with channels for conductors along the lead body 102. In one or more examples, the spline 120 may be disposed at the lead proximal end 108.

In some examples, protrusions 122 and channels 123 extend along the entire length of spline 120. In some examples, splines 120 defines a plurality of separate spline segments and protrusions 122 and channels 123 may be defined on each of the spline segments. Longitudinally adjacent separate spline segments may be separated by a longitudinal gap. For example, in some examples, the separate spline segments may be completely separated and independent from other spline segments. In some examples, separate spline segments may be connected via a reduced-diameter segment extending across the longitudinal gaps. These longitudinal gaps may provide various advantages. For example, when injection molding the outer jacket of the lead around the spline, pressure from m the flowing polymer may cause the adjacent protrusions to fold over. If there are longitudinal gaps between one or more protrusions, the injection molding material can be injected at one or more of these longitudinal gaps to reduce the deformation of any splines.

In one or more examples, the spline may define a stylet lumen 120 therein, where the stylet lumen 120 may be sized and configured to receive a stylet therethrough. In some examples, the spline 118 may have a predetermined hardness. In one or more examples, the spline 118 may have a first hardness at the spline proximal end and a second hardness at the spline distal end, where the first hardness may be different than the second hardness. In some examples, the first hardness may be greater than the second hardness. In some examples, the second hardness may be greater than the first hardness. In some examples, the spline 118 may have a 55D stiffness.

A manufacturing system may insert conductors 114 within channels 123 of spline 118. The manufacturing system may advance conductors 114 may longitudinally through channels 123 or insert conductors 114 radially into channels 123. The manufacturing system may position contacts 112 radially over spline 118 and conductors 114 and electrically couple contacts 112 to respective conductors 114 (e.g., via welding, a mechanical coupling feature, an adhesive). The manufacturing system may form the outer lead body of lead body 102 over the assembled lead (without the outer lead body). In some examples, the outer lead body may be injection molded or overmolded over conductors. For instance, the manufacturing system may place the assembled lead into a mold form. The manufacturing system may then begin to introduce (e.g., via injection) a biocompatible polymer or other biocompatible material into the mold form to form outer lead body. The injected material may surround any exposed portions of conductors 114, spline 118, welds, other and lead structures, but may not cover the external portions of contacts 112. The manufacturing system may introduce the material into the mold form via one or more mold gates in the mold form. The mold gates may be disposed between longitudinally adjacent contacts 112 and, in some examples where spline 118 defines a plurality of separate spline segments (e.g., protrusions separated by one or more longitudinal gaps), between longitudinally adjacent spline segments. As discussed herein, mold gates at these longitudinal gaps or between spline segments may reduce the likelihood that the flowing material through the mold gate will push over or otherwise deform a portion of the protrusion(s) adjacent the mold gate.

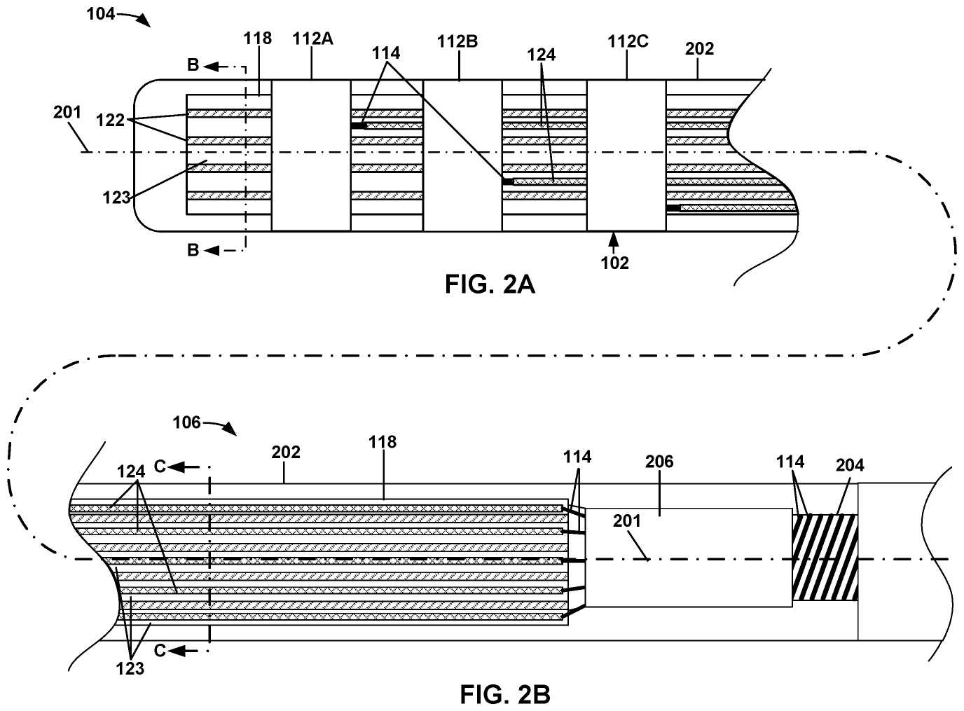

FIG. 2A is a partial cross-section of an example of the lead 100 of FIG. 1A at the first end 104 of lead 100. FIG. 2B is a partial cross-section of the example lead 100 of FIG. 1A at medial portion 106 of lead 100. The cross-sections illustrated in FIGS. 2A and 2B are taken along a reference plane extending along the longitudinal length of lead 100. First lead end 104 of lead 100, alternatively referred herein to as “lead distal end 104” or “distal end 104,” may contact tissue of a patient. Second end 108 of lead 100, alternatively referred to herein as “lead proximal end 108” or “proximal end 108,” may be coupled to an implantable medical device (IMD), a medical system, or the like.

As illustrated in FIG. 2A, lead distal end 104 may include spline 118 disposed within an outer lead body 202 and a plurality of contacts 112 disposed radially over spline 118 and outer lead body 202. Spline 118 may include a plurality of protrusions 122 defining a plurality of channels 123. Conductors 114 may be disposed within channels 123 and each of conductors 114 may be electrically coupled to a corresponding contact of the plurality of contacts 112. One or more of conductors 114 may be at least partially disposed within one or more slip tubes 124 in some examples. While FIG. 2A primarily illustrates and describes contacts 112 in terms of ring electrodes, other types of electrical contacts may be included in an example lead 100.

Lead 100 may be described as including a complex electrode array geometry. A complex electrode array geometry may be an electrode array that includes at least one level of segmented ring electrodes (e.g., circumferentially positioned electrodes). In another example, a complex electrode array geometry may refer to an electrode array that includes electrodes centered in two, three, or even more planes. A complex electrode geometry may indicate any electrode array in which different electrode combinations may be used to deliver electrical stimulation in multiple directions away from the lead. Thus, the complex electrode array geometry may include multiple levels of segmented ring electrodes, segmented ring electrodes and ring electrodes, or any other combination of electrodes including at least one level of segmented ring electrodes. Segmented ring electrodes may generally be two or more electrodes located at different angular or circumferential positions around the circumference of lead body 102. Segmented ring electrodes or other complex electrode array geometries may be used to produce customizable stimulation fields (e.g., electrical fields that may affect or activate patient tissue) that may be directed to a particular side of lead 100 in order to isolate the stimulation field around the target anatomical region of a brain in DBS, for example.

Lead 100 may have any suitable configuration. Lead body 102 may be substantially cylindrical (e.g., cylindrical or nearly cylindrical) in shape (e.g., may have a circular or nearly circular cross-section when the cross-section is taken in a direction perpendicular to a longitudinal axis 201 of lead 100). In other examples, however, lead 100 may have another suitable shape. For example, lead 100 may define one or more curves, e.g., a shape configured to reach target anatomical regions of the patient. In some examples, lead 100 may be similar to a flat paddle lead or a conformable lead shaped for the patient. Also, in other examples, lead 100 may for constructed of any of a variety of different polygonal cross sections taken transverse to the longitudinal axis of the lead. Although lead 100 may be generally flexible, a lead may include one or more portions that are semi-rigid or rigid to aid in implantation and/or achieve desired orientation of lead 100 within the patient.

Outer lead body 202 may be formed from an insulative biocompatible material. Example biocompatible materials may include at least one of polyurethane, silicone, and fluoropolymers such as TFE, PTFE, and/or expanded PTFE (i.e., porous ePTFE, nonporous ePTFE). Outer lead body 202 may be a molded lead body that at least partially surrounds a lead structure that supports contacts 112, conductors 113, and spline 118. In some examples, outer lead body 216 may be injection molded.

Within outer lead body 202, lead 100 includes spline 118. Spline 118 may extend along longitudinal axis 210 in distal end 104. Spline 118 may define two or more protrusions 122 extend radially away from longitudinal axis 201 and from an outer surface of spline 118 and defining two or more channels 123 within spline 118. The outer surface of spline 118 may be defined by the apices of channels 123. Each of channels 123 may be electrically isolated from a circumferentially adjacent channel 123 via protrusions 122. At least one of protrusions 122 may define a maximum outer radius of spline 118. A distance between the radius of an inner surface of one or more of contacts 112 and the maximum outer radius of spline 118 may be less than a diameter of one or more of conductors 114, e.g., to inhibit travel of conductors 114 out of channels 123. Protrusions 122 may extend away from the outer surface of spline 118 by a uniform distance or by a varying distance, e.g., to stability spline 118 within contacts 112 and/or to facilitate assembly of lead 100. Protrusions 122 may define uniform or varying shapes, e.g., to facilitate retention of conductors 114 within channels 123. When a manufacturing system positions spline 118 and conductors 114 within one of contacts 112, one or more protrusions 122 may contact an inner surface of the contact 122 and cause spline 118 to maintain a relative radial position within the contact 112. Protrusions 122 may all have the same radial height or one or more of protrusions 112 may differ in radial height in other examples. Differences in radial height may reduce friction when sliding electrodes or other components over spline 118 while still maintaining electrical insulation between conductors 114.

Conductors 114 may be disposed within channels 123 of spline 108. Conductors 114 may transmit electrical signals along the length of conductors 114 between contacts 112 and a second set of contacts disposed on proximal end 108. Each of conductors 114 may be electrically insulated and electrically isolated from other conductors 114. Lead 100 may include one, two, or three or more conductors 114. In some examples, such as when conductors 114 are configured to extend along longitudinal axis 210, at least a portion of conductors 114 may be disposed within corresponding slip tubes 124. The slip tubes 124 may increase flexure capability of conductors 114 relative to an identical conductor 114 that is not disposed within a slip tube 124.

As illustrated in FIG. 2B, spline 118 may extend at least partially into medial portion 106 of lead 100. A second end (e.g., a proximal end) of spline body of spline 118 may terminate in or distal to medial portion 106. Conductors 114 may extend from proximal end 108 towards distal end 104 along and around an elongated body 204 within lead body 102. For example, conductors 114 may define one or more helices, spirals, or coils around elongated body 204 and along longitudinal axis 201. Elongated body 204 may define an inner lumen in fluid communication with stylet lumen 120 of spline 118 and similarly sized and configured to receive a stylet therethrough. Conductors 114 may be electrically insulated and/or electrically isolated from one another.

Conductors 114 may extend into a structure 206 (e.g., a tubing 206) which aligns conductors 114 with corresponding channels 123 on spline 118. In some examples, one or more conductors 114 may be configured to be disposed within specific channels 123 on spline 118. In some examples, each of conductors 114 may be disposed within any of channels 123 on spline 118. Conductors 114 may be inserted into slip tubes 124 at or around a proximal end of spline 118. Once conductors 114 are disposed within channels 123, conductors 114 extend along the respective channels 123 until the distal ends of conductors 114 longitudinally overlap with the respective contacts 112. The distal ends of conductors 114 may be electrically coupled with the respective contacts 112. The distal ends of conductors 114 may be exposed from slip tubes 124 to facilitate fixation of conductors 114 to contacts 112.

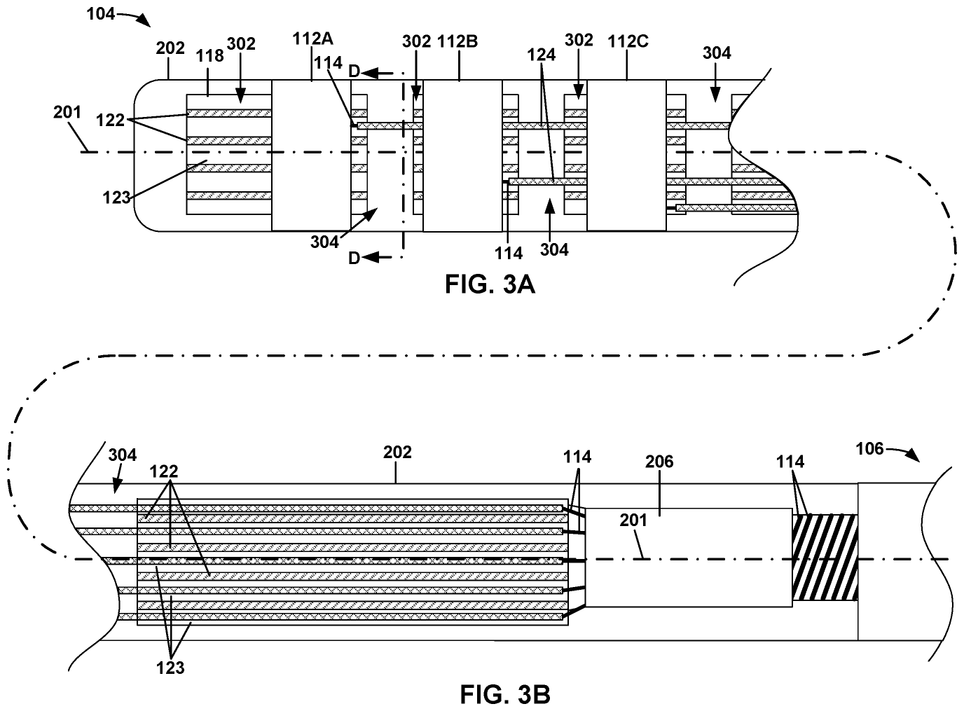

FIG. 3A is a partial cross-section of another example of lead 100 of FIG. 1A at the distal end 104 of lead 100. FIG. 3B is a partial cross-section of the example lead 100 of FIG. 3A along a medial portion 106 of lead 100. As compared to the example illustrated in FIGS. 2A and 2B, the example of lead 100 of FIGS. 3A and 3B illustrates spline 118 include a plurality of separate spline segments 302 separated by longitudinal gaps 304. Each of spline segments 302 may define protrusions 122, channels 123, stylet lumen 120, and any other features of spline 118 as described herein. Protrusions 122, channels 123 and stylet lumen 120 of spline segments 302 may be circumferentially aligned such that conductors 114, slip tube 124, and/or stylets may extend across multiple spline segments 302 in a same manner as a spline 118 with a continuous spline body, e.g., as illustrated in FIGS. 2A and 2B.

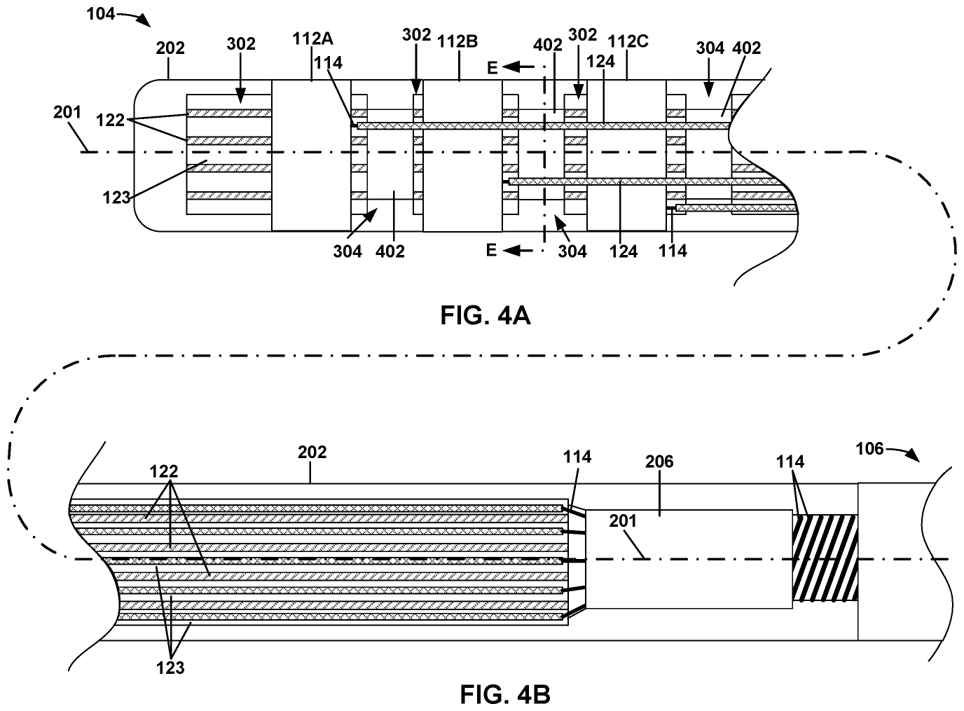

Spline segments 302 may maintain a consistent location and orientation within lead body 102 due to confinement of spline segments 302 by outer lead body 202 of lead 100. A manufacturing system may flow a biocompatible polymer or other biocompatible material around and over spline segments 302 and into longitudinal gaps 304 to retain spline segments 302 within lead body 202 at specific orientations and positions. Spline segments 302 may be positioned radially within contacts 112, e.g., to facilitate coupling of conductors 114 to contacts 112. Spline segments 302 may be longer than contacts 112 along longitudinal axis 201. Longitudinal gaps 304 may be defined by spline segments 302 that are completely separate from each other, or a single spline that has a continuous core or spline body in which one or more protrusions running the length of the spline are segmented to define the longitudinal gaps. Separate spline segments 302 may provide several advantages over a continuous spline. The reduced length of the separate spline segments 302 may facilitate assembly of contacts 112, conductors 114, and spline 118 prior to the introduction of a material to form outer lead body 202 around the assembled components by reducing interference between portions of spline 118 (i.e., spline segments 302) and inner surfaces of contacts 112. In some examples, where the manufacturing system forms outer lead body 202 via an injection molding or overmolding technique, mold gates may be disposed within longitudinal gaps 304 between spline segments 302. Placement of mold gates within longitudinal gaps 304 may reduce a likelihood and/or effect of deformation of spline 118 (e.g., of protrusions 122) as a result of the pressurized introduction of the material into a mold form containing the assembled contacts, conductors 114 and spline 118. Deformation of protrusions 122 may reduce electrical insulation and/or electrical isolation capabilities of spline 118 and/or lead to separation of spline 118 from electrical contacts 112. The use of separate spline segments 302 may reduce and/or prevent deformation of protrusions 122 on spline 118 between contacts 112 as the introduction of the material would not cause the material to apply forces to protrusions 122 along reference axes orthogonal to longitudinal axis 201 and cause deformation of protrusions 122. Alternatively, a continuous spline 118 in which one or more of the protrusions are segmented along the length of the spline to create the longitudinal gaps between protrusion segments at the same circumferential position. Such an example may be similar to the example of FIG. 4A.

FIG. 4A is a partial cross-section of another example of lead 100 of FIG. 1A at distal end 104 of lead 100. FIG. 4B is a partial cross-section of the example lead 100 of FIG. 4A along a medial portion 106 of lead 100. As illustrated in FIGS. 4A and 4B, separate spline segments 302 may be connected across longitudinal gaps 304 via ablated segments 402. Ablated segments 402 may define a reduced diameter section of spline 118. Ablated segments 402 may define an outer diameter less than, equal to, or greater than the diameter of the outer surface of spline 118 (e.g., as defined by apices of channels 123). Ablated segments 402 may define a relatively smooth outer surface, as illustrated in FIG. 4A, or may define protrusions extending partially away from longitudinal axis 201 to a distance less than protrusions 122.

A manufacturing system may form separate spline segments 302 and ablated segments 402 by removing material from a monolithic, or continuous, spline 118 until ablated segments 402 define a specified diameter and/or length. The specified diameter may be about the same as a minor diameter of spline 118. In some examples, the length of ablated segments 402 along longitudinal axis 201 may be about 0.051 centimeters (cm) (e.g., about 0.02 inches (in)). The manufacturing system may remove material via ablation (e.g., laser ablation) of portions of spline 118 corresponding to ablated segments 402 or via one or more means of removing material different from ablation including, but are not limited to, laser etching, chemical etching, or via a mechanical cutting implement. In some examples, the manufacturing system may remove material from separate spline segments 302 to define curvature, taper, or chamfer or protrusions 122 at the edges of spline segments 302 abutting ablated segments 402. The curvature, taper, or chamfer of edges of protrusions 122 to facilitate insertion of spline segments 302 into contacts 122 or placement of contacts 112 over spline segments 302. Spline segments 302 and ablated segments 402 may be arranged along the length of spline 118 to coincide with the placement of contacts 112, such that contacts 122 longitudinally overlap with spline segments 302. For example, each spline segment 302 may have a longitudinally length at least equal to the longitudinal length of a corresponding contact 112 and ablated segments 402 may define longitudinal lengths less than or equal to a longitudinal length of longitudinal gaps 304 between adjacent spline segments 302.

Ablated segments 402 may improve alignment of channels 123 and of stylet lumens 120 of spline segments 302. The reduced diameter of ablated segments 402 may reduce a likelihood and/or magnitude of deformation of spline 118 due to the introduction of material around stylet 118 from mold gates. The reduced diameter of ablated segments 402 may facilitate advancement of contacts 112 along longitudinal axis 210 and over spline 118 by reducing an amount of interference between spline 118 and contacts 112. Although ablated segments 402 show that all protrusions around the spline 118 are missing protrusion features at this location, one or more protrusions may still run completely through this region. In this manner, the longitudinal gaps may refer to longitudinal gaps in at least one protrusion of spline 118, but not all protrusions, in some examples.

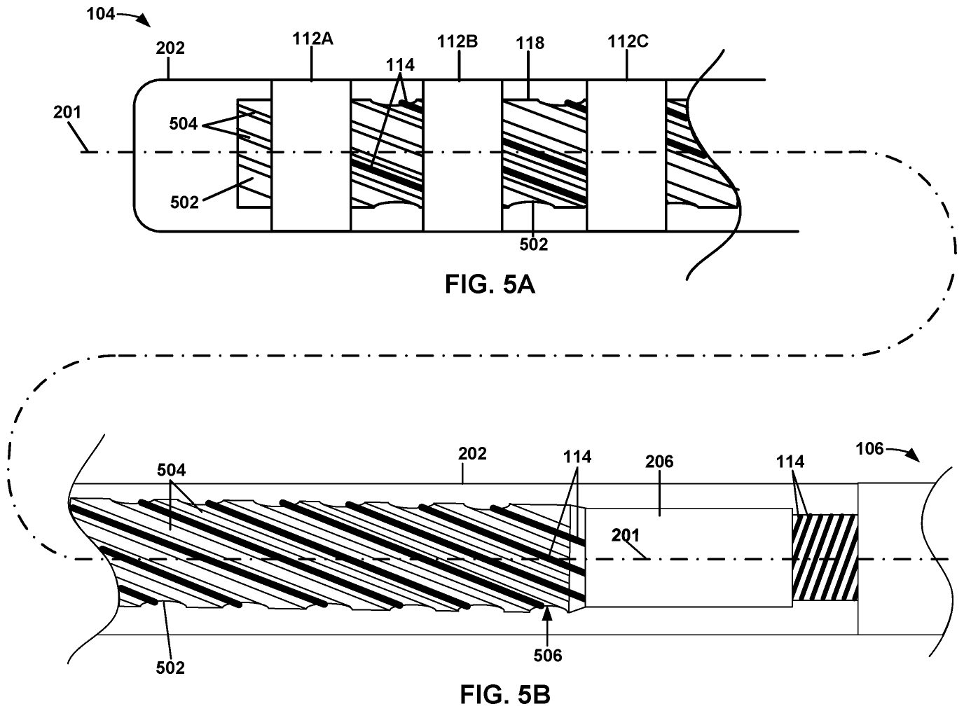

FIG. 5A is a partial cross-section of another example of lead 100 of FIG. 1A at distal end 104 of lead 100. FIG. 5B is a partial cross-section of the example lead 100 of FIG. 5A along a medial portion 106 of lead 100. As illustrated in FIGS. 5A and 5B, spline 118 includes a plurality of protrusions 504 defining channels 502, each channel 502 being configured to receive a conductor 114. Plurality of protrusions 504 may define similar dimensions (e.g., heights, widths) as protrusions 122 and may define helices or spirals along and around longitudinal axis 201. For example, spline 118, as illustrated in FIGS. 5A and 5B, may define a substantially similar or same cross-section as one or more other example configurations of spline 118 described herein, e.g., as illustrated in FIGS. 1-4B. Protrusions 504 may define channels 502 in helices or spirals along and around longitudinal axis 201. Helices or Spirals described herein may extend along a reference helical axis and may define less than one, one, or more than one full revolution around the perimeter of spline 118.

The helices or spirals defined by protrusions 504 and channels 502 may be wound in a clockwise or counterclockwise direction around longitudinal axis 201. The helices or spirals may extend along the entire length of spline 118 from first lead end 104 to proximal end 506 of spline 118. In some examples, the helices or spirals extend partially but not entirely along the length of spline 118. For example, protrusions 504 and channels 502 may define helices or spirals extending from proximal end 506 and towards first lead end 104. In such examples, protrusions and channels along portions of spline 118 not containing helices or spirals may define any of the other examples spline configurations described within this disclosure, e.g., as illustrated in FIGS. 1-4B.

Protrusions 504 and channels 502 may define one or more loops defining the helices or spirals around the outer perimeter of spline 118. Adjacent loops may be separated along longitudinal axis 201 by a pitch. The pitch between adjacent loops may be uniform or may be varied. For example, protrusions 504 and channels 502 at or around first lead end 104 may define a smaller pitch than protrusions 504 and channels 502 at or around proximal end 506 of spline 118. Protrusions 504 may define between about 1.5 revolutions per inch (e.g., about 0.667 inches per revolution) to about and about 3 revolutions per inch (e.g., about 0.333 inches per revolution).

The one or more loops may each be defined by a helical angle defining an offset of each loop (e.g., relative to a reference plane coincidental to or orthogonal to longitudinal axis 201. The helical angle may be uniform or varied along the length of spline 118. In some examples, the helical angle may be about 50 degrees to about 75 degrees relative to longitudinal axis 201.

Channels 502 may cause conductors 114 to extend at least partially around the outer perimeter of spline 118. Such a configuration may increase the flexibility of conductors 114 without the use of slip tubes 124. In some examples, conductors 114 within channels 502 and without slip tubes 124 may exhibit a same level of flexibility as identical conductors 114 within channels 123 and with slip tubes 124.

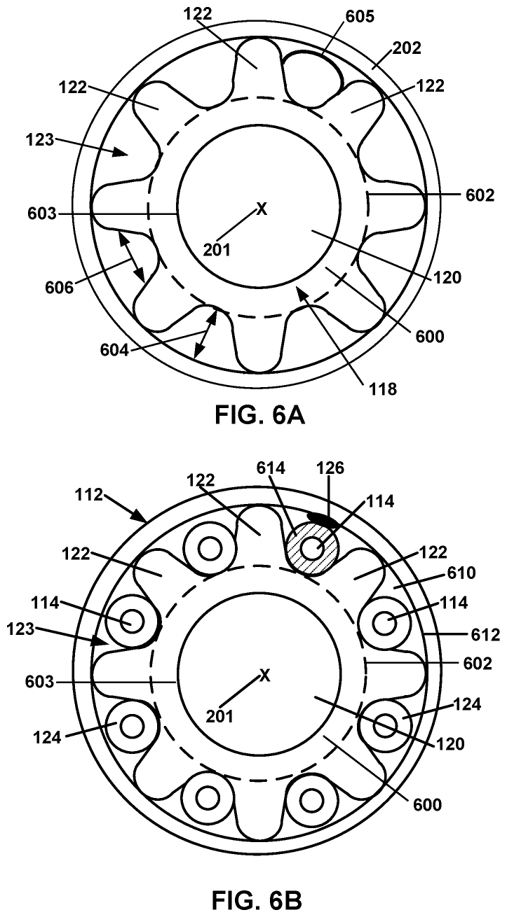

FIG. 6A is a cross-section of the lead of FIG. 2A along line B-B in FIG. 2A. FIG. 6B is a cross-section of the lead of FIG. 2B along line C-C in FIG. 2B. FIG. 6A illustrates an example cross-section of spline 118 at a location without conductors 114 disposed within channels (e.g., channels 123) of spline 118. FIG. 6B illustrates an example cross-section of spline 118 at a location with conductors 114 disposed within channels of spline 118. As illustrated in FIGS. 6A and 6B, Spline 118 may define a spline body 600 extending along longitudinal axis 201 and defining outer surface 602 and inner surface 603. Inner surface 603 may define a stylet lumen 120 extending through spline body 600 from a first end to a second end. Spline 118 may include a plurality of protrusions 122 extending radially away from outer surface 602 of spline body 600. The plurality of protrusions 122 may define channels 123 between circumferentially adjacent protrusions 122. An outer lead body 202 of lead body 102 may be disposed radially over spline 118 (e.g., radially outside of protrusions 122). Outer lead body 102 may contact apices of protrusions 122.

Spline 118 may define protrusions 122 equally distributed around the outer perimeter of spline body 600. Protrusions 122 may define uniform dimensions (e.g., uniform heights, uniform widths) and/or uniform shapes. In some examples, protrusion 122 may define variations in dimensions and/or shapes. Channels 123 between adjacent protrusions 122 may define a height 604 and a width 606 which are substantially the same in this example. Each of height 604 and width 606 may be greater than or equal to a diameter of conductor 114 or of slip tube 124 retaining conductor 114, e.g., to facilitate retention of conductors 114 within channels 123. In other examples, height 604 and width 606 may be slightly less than the diameter of conductor 114 and held in place with injection molding, adhesive, or other mechanism.

Spline 118 may include a plurality of retainers 605 disposed within channels 123. Retainers 605 may facilitate retention of conductors 114 within channels 123 by impeding unintended radially outward movement of conductors 114 out of channels 123 during assembly of lead 100. Retainers 605 may define a ring connecting circumferentially adjacent protrusions 122. Retainers 605 may define a C-shaped ring (e.g., as illustrated in FIG. 6A), an U-shaped ring, a V-shaped ring, or any other shape or configuration that may retain conductors 114. A distance between each retainer 605 and a radially inward-most position within a corresponding channel 123 may be at least equal to an outer diameter of conductor 114 or of slip tube 124. In some examples, retainer 605 may elastically deform in response to the insertion of conductor 114 within channel 123 to facilitate retention of conductor 114 within channel 123.

In some examples, conductor 114 may be inserted into channel 123 radially-inwards of retainers 605. In such examples, conductor 114 is advanced longitudinally along channel 123 and between channel 123, protrusions 122 defining channel 123, and retainer 605. In some examples, conductor 114 may be inserted into channel 123 in a radial direction orthogonal to longitudinal axis 201 and over retainers 605. In such examples, one or more retainers 605 may deform (e.g., elastically, plastically) into channel 123 and retain conductor 114 within channel 123 (e.g., via friction forces between the one or more retainers 605 and conductor 114.

Each channel 123 may include one or more retainers 605 disposed at different longitudinal locations along longitudinal axis 201. Retainers 605 for each channel 123 may be disposed proximal to a specified position for contact 112 corresponding to conductor 114 configured to be disposed within the each channel 123. A manufacturing system may form retainers 605 as a part of forming spline body 600 or may affix retainers 605 to spline 118 prior to insertion of conductors 114 into channels 123 of spline 118.

As illustrated in FIG. 6B, conductors 114 and/or slip tubes 124 may be disposed within channels 123. When conductors 114 and/or slip tubes 124 are disposed within channels 123, conductors 114 and/or slip tubes 124 may not extend radially past apices of protrusions 122. As illustrated in FIG. 6B when contact 112 is disposed radially over spline 118 (i.e., when spline 118 is disposed radially within an inner lumen 610 of contact 112), one or more of protrusions 122 may contact inner surface 612 of contact 112.

Spline 118 may define any number of protrusions 122 and channels 123. The number of protrusions 122 and channels 123 may be based on the number of contacts 112 disposed on lead 100. For example, spline 118 may define a same number of channels 123 as the number of contacts 112. Spline 118 may include less than eight, eight, or more than eight protrusions 122 and/or less than eight, eight, or more than eight channels 123. Each channel 123 may be configured to retain a conductor 114 or may be configured to isolate adjacent channels 123.

In some examples, as illustrated in FIG. 6B, conductors 114 may be disposed within a crimp sleeve 614. Crimp sleeve 614 may be positioned along the longitudinal length of conductor 114 and may be distal to slip tube 124. In such examples, crimp sleeve 614 and slip tube 124 may define a uniform outer diameter of conductor 114 along longitudinal axis 210. In some examples, at least a portion of crimp sleeve 614 may be disposed within or over slip tube 124. Crimp sleeve 614 may be welded (e.g., laser-welded) to inner surface 612 of contact 112 at position 126. Crimp sleeve 614 may electrically couple conductor 114 within crimp sleeve 614 to contact 112 via the weld at position 126.

Channels 123 within spline 118 may be size to facilitate longitudinal movement of conductors 114 and crimp sleeves 612 through channels 123. In some examples, circumferentially adjacent protrusions 122 are separated by a minimum distance greater than or equal to the outer diameter of crimp sleeve 614 containing conductor 114. The separation of circumferentially adjacent protrusions 122 by the minimum distance may facilitate advancement or retraction of crimp sleeves 612 and conductors 114 within channels 123 of spline 118 (e.g., by a manufacturing system) to longitudinally align each crimp sleeve 614 with a corresponding contact 112.

In some examples, circumferentially adjacent protrusions 122 are separated by a maximum distance and/or a radially inward-most position within channels 123 are separated from the inner surface 612 of contact 112 by the maximum distance. The maximum distance may be about the outer diameter of crimp sleeve 614 to facilitate contact between crimp sleeve 614 and inner surface 612 at position 126 and to facilitate welding of crimp sleeve 614 and inner surface 612 at position 126, e.g., by reducing or eliminating a gap between an outer surface of crimp sleeve 614 and inner surface 612 of contact 112. A maximum gap between an outer surface of crimp sleeve 614 and inner surface 612 at position 126 may be up to about. 051 mm (e.g., about 0.002 in).

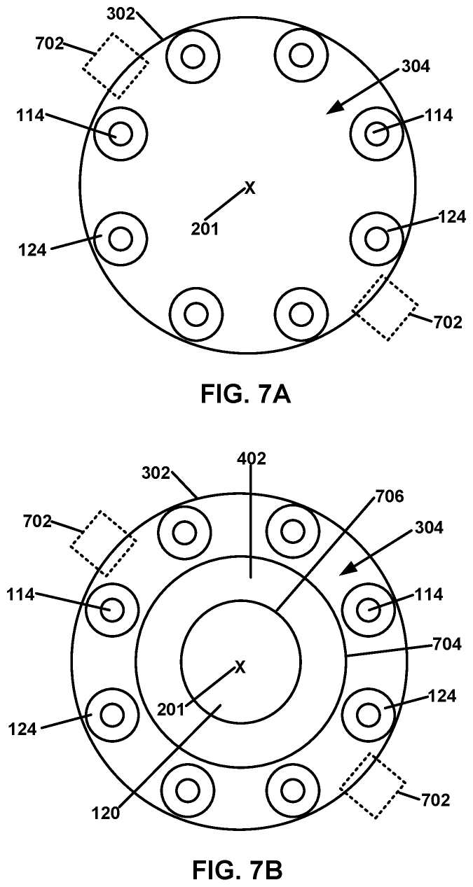

FIG. 7A is a cross-section of the lead of FIG. 3A along line D-D in FIG. 3A. FIG. 7A illustrates an example cross-section at a longitudinal gap 304 of the example spline 118 illustrated in FIGS. 3A and 3B. In some examples, as illustrated in FIGS. 3A and 7A, only conductors 114 and slip tubes 114 may extend across longitudinal gaps 304 between adjacent spline segments 302. During manufacture of lead 100, mold gates 702 may be disposed within longitudinal gaps 304. Placement of mold gates 702 within longitudinal gaps 304 may reduce or inhibit deformation of protrusions 122 resulting from influx of moldable material around spline 118 during an injection molding or overmolding process. Mold gates 702 may be place along the outer perimeter of lead 100 and at opposing locations to provide for uniform injection of material around spline 118. An example manufacturing system may include one, two, or three or more mold gates 702 at one or more of longitudinal gaps 304. Mold gates 702 may be disposed within one or more of longitudinal gaps 304 or may be disposed at each longitudinal gap 304. Mold gates 702 within different longitudinal gaps 304 may be arranged at same or different orientations (e.g., may be circumferentially aligned, may be circumferentially offset).

FIG. 7B is a cross-section of the lead of FIG. 4A along line E-E in FIG. 4A. FIG. 7B illustrates an example cross-section of ablated segments 402 of the example spline 118 illustrated in FIG. 4A. In some examples, as illustrated in FIGS. 4A, 4B, and 7B, separate spline segments 302 are connected across longitudinal gaps 304 by ablated segments 402. Each of ablated segments 402 may define an outer surface 704 and an inner surface 706. Inner surface 706 may define a portion of style lumen extending through the respective ablated segment 402. Outer surface 704 may be relatively smooth and may be devoid of protrusions 122. Outer surface 704 may define a diameter less than, equal to, or greater than the diameter of outer surface 602 of spline body 600. A manufacturing system may form outer surface 704 be removing protrusions 122 and material from spline body 600 from the respective ablated segment 402 until the outer diameter of outer surface 704 is less than a threshold diameter. As illustrated in FIG. 7B, mold gates 702 are disposed within longitudinal gap 304 and may be used to introduce a moldable material around spline 118. Ablated segments 304 may maintain separated spline segments 302 as a single unitary body. Ablated segments 304 may reduce the risk of deformation of protrusion 122 during an injection molding or overmolding process due to the absence of protrusions 122 along ablated segments 304. In other examples, one or more of protrusions 122 may still remain in this section of longitudinal gaps 304 where a portion of at least one protrusion has been removed (or not formed) to create the longitudinal gap.

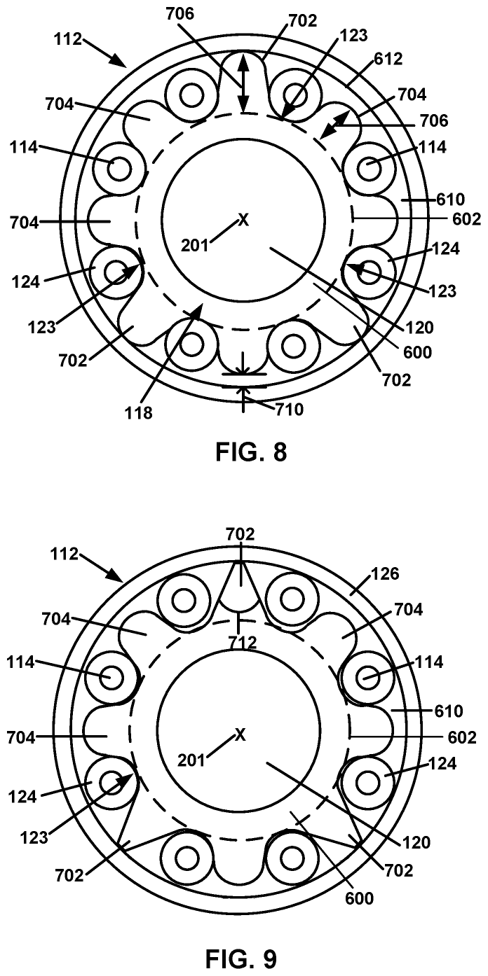

FIG. 8 is another example cross-section of the lead of FIG. 2A along line B-B in FIG. 2A. FIG. 9 is another example cross-section of the lead of FIG. 2A along line B-B in FIG. 2A. In some examples, as illustrated in FIGS. 8 and 9, protrusions may extend away from outer surface 602 of spline body 600 for different radial distances, thereby defining varying heights of the protrusions. Inclusion of protrusions of varying heights around spline 118 may facilitate assembly of lead 100 (e.g., insertion of spline 118 into inner lumen 610 of contacts 112) by reducing the points of contact, and thereby friction, between contacts 112 and spline 118. Protrusions 702 and 704 may be formed with these dimensions, or one or more equally formed protrusions may have material removed (e.g., stripped, ablated, etc.) in order to create protrusions with different radial heights and/or shapes.

In the example spline 118 illustrated in FIG. 8, a plurality of protrusions 702 and 704 are disposed around the outer perimeter of spline body 600, each of protrusions 702 and protrusions 704 extending radially away from longitudinal axis 201. Protrusions 702 may extend away from longitudinal axis 201 by a first distance and may define a first height 706. Protrusions 704 may extend away from longitudinal axis 201 by a second distance and may define a second height 708. When spline 118 is inserted within inner lumen 610 of contact 112, protrusions 702 may contact inner surface 612 of contact 112 and protrusions 704 may be separated from inner surface 612 by a distance 710. First height 706 may be equal to a sum of second height 708 and distance 710. Separation of protrusions 704 from inner surface 612 may reduce friction and/or resistance to insertion of spline 118 within inner lumen 610 and/or advancement of contact 112 over spline 118, thereby facilitating placement of contact 112 over spline 118. Distance 710 may be less than an outer diameter of conductor 114 and/or of slip tube 124, e.g., to inhibit travel of conductor 114 from within one channel 123 to another channel 123.

Protrusions 702 may interface with (e.g., contact) inner surface 610 of contacts 112 to inhibit unintended movement of contact 112 relative to spline (e.g., during assembly, during molding of lead body 102, during navigation within the body of a patient). Protrusions 702 may be equally spaced around the outer perimeter of spline 118 to increase the stability of the interface between protrusions 702 and contact 112. Spline 118 may include two, three, or four or more protrusions 702 disposed around the outer perimeter of spline body 600. Spline may include protrusions 704 distributed between adjacent protrusions 702, e.g., such that protrusions 702 and 704 are equally spaced around the outer perimeter of spline body 600 and define a plurality of equally spaced channels 123.

Protrusions 702, 704 may define similar or varied shapes. For example, as illustrated in FIG. 8, protrusions 702, 704 define elliptical or semi-cylindrical cross-sections and define curved surfaces at apices of the respective protrusions. In some examples, as illustrated in FIG. 9, protrusions 702 define triangular or quadrilateral cross-sections and define pointed or flat surfaces at the apices of the protrusions. The different cross-sections may improve retention of conductors 114 and/or slip tubes 124 within channels 123 and/or reduce resistance to insertion of spline 118 into inner lumen 610 of contact 112. Where protrusions 702 define a triangular or quadrilateral cross-section, sides of protrusions 702 may define an angle 712. Angle 712 may indicate a relative steepness of protrusions 702 and the walls of channels 123 defined by protrusions 702. Angle 712 may be increased to improve strength of protrusions 702 and/or increase contact area between protrusions 702 and inner surface 612. Angle 712 may be decreased to further reduce resistance between contact 112 and spline 118 and/or increase a number of protrusions 702, 704, and channels 123 disposed around the outer perimeter of lead body 600.

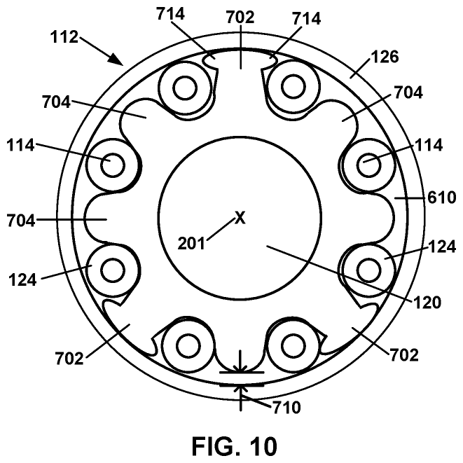

FIG. 10 is another example cross-section of the lead of FIG. 2A along line B-B in FIG. 2A. As illustrated in FIG. 10, protrusions 702 may include fingers 714 extending from a radially distal-most section of protrusions 702. In other words, protrusions 702 may include fingers 714 in order to have a larger cross-sectional width further from the center axis 201 than closer to the center axis 201. Fingers 714 may extend into and/or towards channels 123 and may interface with conductors 114 and/or slip tubes 124 within channels 123 to inhibit unintended movement of conductors 114 and/or slip tubes 124 out of channels 123, e.g., via a snap-fit arrangement. Fingers 714 may, in conjunction with sides of protrusions 702, 704, retain conductors 114 and/or slip tubes 124 within channels 123. For example, fingers 714 may snap around conductors 114 and/or slip tubes 124 to inhibit radially outward movement of conductors 114 and/or slip tubes 124. In some examples protrusions 702 include fingers similar to fingers 714 and being configured to releasably retain conductors 114 and/or slip tubes 124.

While each of the example spline designs in any of FIGS. 2A-10 and the accompanying descriptions are described individually above, an example lead 100 with an example spline 118 may include features from any individual example or any combination of examples described herein. For example, an example spline 118 may include separate spline segments 302 and/or ablated segments 402 at or around a first lead end 104 of lead body 102 but not at locations more proximal to first lead end 104. In some examples, protrusions along spline 118 may helixes or spirals at or around a proximal end of spline 118 and extend along longitudinal axis 201 at or around first lead end 104. In some examples, spline 118 may include any combination of protrusions 122, 504, 702, 704. For example, spline 118 may define a plurality of longitudinal regions, each longitudinal region including protrusions of different shapes, dimensions, and/or arrangements.

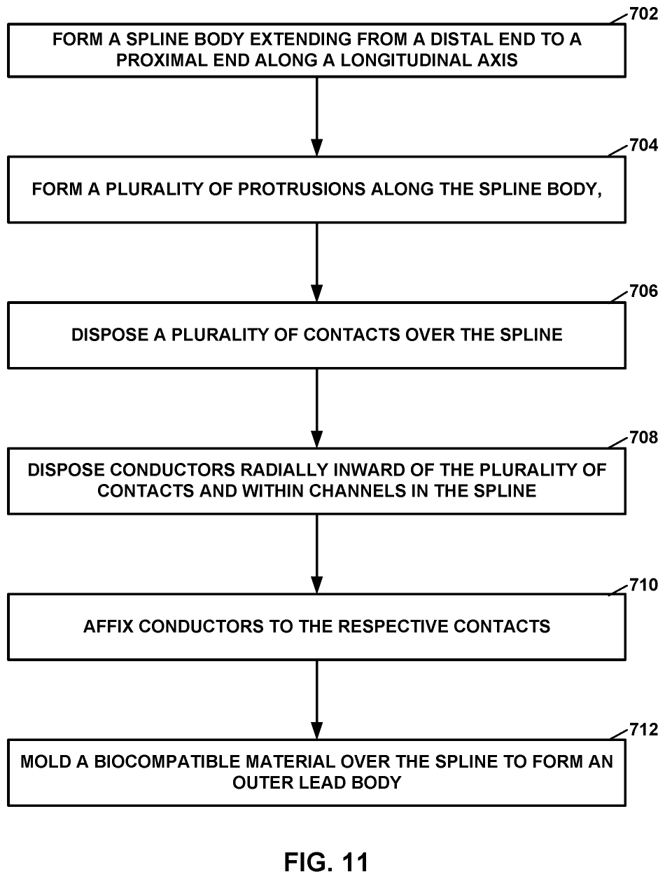

FIG. 11 is a flow chart illustrating an example process of manufacturing a medical lead 100, in accordance with the examples described in this disclosure. While FIG. 11 is primarily described herein with respect to the example leads 100 illustrated in FIGS. 1-10, the same process may be applied to manufacture any medical lead described herein. Additionally, the example process described herein may be performed by a manufacturer, a manufacturing system, and/or one or more manufacturing devices.

A manufacturing system may form a spline body (e.g., spline body 600) of a spline 118 extending from a distal end to a proximal end along a longitudinal axis (e.g., longitudinal axis 201) (702). The manufacturing system may form spline body 600 as an elongated body. The manufacturing system may form spline body 600 from one or more biocompatible materials including, but are not limited to, polyurethane (e.g., Pellethane, elasthane, carbothane) nylon, polyetheretherketone (PEEK), or any other extrudable semi-rigid biocompatible polymer. The biocompatible material may be compatible with and/or may bond to another biocompatible material used to form the outer lead body 102. The manufacturing system may form a stylet lumen 120 through spline body 600 from the distal end to the proximal end.

The manufacturing system may form a plurality of protrusions (e.g., protrusions 122, 504, 702, 704) along spline body 600 (704). Protrusions may extend from outer surface 602 of spline body 600 radially away from longitudinal axis 201. Protrusions may be equally distributed around the outer perimeter of spline body 600 in some examples, or asymmetrically distributed in other examples. Adjacent protrusions may define channels (e.g., channels 123, 502). For example, the walls of adjacent protrusions may define a channel between the adjacent protrusions. Protrusions may extend at least partially along the longitudinal length of spline body 600. Protrusions may define one or more cross-sectional shapes including, but are not limited to, elliptical, semi-cylindrical, triangular, rectangular, or any other geometric-shaped cross-sections. In some examples, spline body 600 may include protrusions of a first shape, dimensions, or arrangement along a first region and protrusions of a second shape, dimensions, or arrangements along a second region. For example, spline 118 may include protrusions extending along longitudinal axis 201 in a first region and protrusions extending around along and around longitudinal axis 201 to define one or more helices or spirals in a second region different from the first region. In some examples, some of protrusions may extend a greater distance from outer surface 602 of spline body 600 than other protrusions extending from the same spline body 600, e.g., to facilitate placement of contacts 112 over spline body 600. In some examples, the manufacturing system may form retainers 605 within channels 123 and connecting circumferentially adjacent protrusions as a part of forming the plurality of protrusions. In some examples, the manufacturing may affix retainers 605 within protrusions separately and prior to placement of contacts 112 over spline 118 and/or placement of conductors 114 within channels of spline 118.

The manufacturing system may form the plurality of protrusions via one or more manufacturing techniques including, but are not limited to, ablation of spline body 600, removal of material form spline body 600 (e.g., via a cutting instruments), additive manufacturing, three dimensional (3D) printing, or the like.

In some examples, the manufacturing system may define a plurality of separate spline segments 302 along spline body 600. Separate spline segments 302 may be separated by longitudinal gaps 304. Each of the separate spline segments 302 may define protrusions and channels. The protrusions and channels on the separate spline segments 302 may be circumferentially aligned with protrusions and channels on adjacent spline segments 302.

Spline segments 302 may be completely separated, e.g., as illustrated in FIGS. 3A and 3B, or may be connected by ablated segments 402, e.g., as illustrated in FIGS. 4A and 4B. The manufacturing system may completely separate portions of spline body 600 to define spline segments 302 by removing material from spline body 600 via ablation, a heat source, a cutting instrument, or other known techniques. Ablated segments 402 may not define protrusions and channels and define an outer diameter less than, equal to, or greater than the outer diameter along an outer surface 602 of spline body 600. The manufacturing system may form ablated segments 402 between spline segments 302 by removing material from spline body 600 (e.g., via ablation techniques, via a cutting instrument) until an outer diameter of each ablated segment 402 is less than or equal to a threshold outer diameter. In some examples, the process of forming the spline body may be the same process that also forms the protrusions, such as a single extrusion or molding process for the spline.

The manufacturing system may then dispose a plurality of contacts 112 over spline 118 (706). Contacts 112 may include, but are not limited to, ring electrodes, cuff electrodes, paddle electrodes, or segmented ring electrodes. Contacts 112 may be directly disposed on and radially outwards of spline 118 (e.g., on top of protrusions). In some examples, such as when each of contacts 112 define an annulus, The manufacturing system may advance contacts 112 along spline body 600 until the manufacturing system determines that contacts 112 are disposed at specific locations along the length of spline 118. In some examples, where spline 118 includes a plurality of separate spline segments 302, each of contacts 112 may disposed radially over one of spline segments 302. Contacts 112 may define an inner surface 612 defining an inner lumen 610. Inner surface 612 may define an inner diameter equal to or similar to the outer diameter of spline body 600, as defined by one or more of protrusions. When contacts 112 are disposed over spline 118, inner surface 612 may interface with one or more protrusions extending from spline body 600 and inhibit unintended movement of contact 112 relative to spline 118.

The manufacturing system may dispose conductors 114 radially inwards of the plurality of contacts 112 and within channels in spline 118 (708). Conductors 114 may be disposed within slip tubes 124, e.g., to increase flexibility of conductors 114 within lead 100. Each of channels 123 may be sized to retain conductors 114 and/or slip tubes 124. The manufacturing system may insert each conductor 114 into a corresponding channel 123 of spline 118 and advance conductor 114 within channel 123 until a distal tip of conductor 114 longitudinally overlaps with a corresponding contact 112. In some examples, where contacts 112 are disposed at different longitudinal levels along lead body 102, conductors 114 may extend different distances along spline 118 to overlap with the corresponding contact 112. In other examples, conductors 114 may be placed along spline 118 prior to adding contacts or electrodes over the spline.

The manufacturing system may affix conductors 114 to the respective contacts 112 (710). The manufacturing system may weld, solder, or otherwise physically and electrically affix each conductor 114 to inner surface 612 of a corresponding contact 112 Conductors 114 may transmit electrical signals between contacts 112 and a therapy device and/or implantable medical device.

The manufacturing system may mold a biocompatible material over spline 118 to form outer lead body 102 (712). The manufacturing system may injection mold or overmold the biocompatible material over spline 118 to encapsulate spline 118 and define an outer lead body 102. The manufacturing system may place a spline assembly (i.e., spline 118, contacts 112, and conductors 114) into a mold form. The manufacturing system may then introduce the biocompatible material into the mold form and around the spline assembly via a plurality of mold gates 611 in the mold form. Once the biocompatible material solidifies, the manufacturing system may remove the formed lead body 102 out of the mold form and finalize or finish the lead body to form a complete lead 100.

Mold gates 611 may be disposed between adjacent protrusions and/or within longitudinal gaps 304 along spline 118 (e.g., between separate spline segments 302 or gaps between protrusion segments). When the manufacturing system introduces the biocompatible material into the mold form through mold gates 611, the absence of protrusions in longitudinal gaps 304 and/or on ablated segments 402 may reduce the likelihood and magnitude of deformation to protrusions resulting from pressure applied on the protrusions form the flow of biocompatible material. The outer lead body 102 may be formed around and/or partially over portions of contacts 112 (e.g., to form electrically insulated regions on contacts 112). Stylet lumen 120 may be maintained by spline 118 and may facilitate the insertion of a stylet into lead 100 to navigate lead 100 within the body of the patient.

The following examples are described herein.

Example 1: a lead assembly comprising: a plurality of conductors, each conductor of the plurality of conductors extending from a conductor first end to a conductor second end along a longitudinal axis; a spline extending from a spline first end to a spline second end, the spline comprising: a spline body extending along the longitudinal axis, and a plurality of projections disposed around an outer perimeter of the spline body, the plurality of projections extending radially away from an outer surface of the spline body, wherein one or more projections of the plurality of projections extend along the longitudinal axis, and wherein each projection of the one or more projections comprises two or more projection segments separated by one or more longitudinal gaps, wherein the plurality of projections define a plurality of conductor channels around the outer perimeter of the spline body, wherein each conductor of the plurality of conductors is disposed within a respective conductor channel of the plurality of conductor channels; a plurality of electrical contacts electrically coupled to respective conductors of the plurality of conductors at the conductor first end; and an outer lead body disposed radially outwards from at least a portion of the spline body and at least a portion of each conductor of the plurality of conductors.

Example 2: the lead assembly of example 1, wherein the plurality of projections are configured to electrically isolate a conductor of the plurality of conductors from a circumferentially adjacent conductor of the plurality of conductors.

Example 3: the lead assembly of any of examples 1 and 2, wherein the plurality of projections define the plurality of conductor channels as one or more helixes extending along a longitudinal length of the spline body and around the outer perimeter of the spline body.

Example 4: the lead assembly of any of examples 1-3, wherein the spline body comprises a plurality of separate spline segments, each separate spline segment comprising: a respective portion of the spline body; and a respective projection segment of the two or more projection segments of each projection of the one or more projections, wherein each spline segment is separated from a longitudinally adjacent spline segment by a respective longitudinal gap, and wherein each separate spline segment longitudinally overlaps with a respective electrical contact of the plurality of electrical contacts.

Example 5: the lead assembly of any of examples 1-4, wherein the plurality of projections comprises: a plurality of first projections extending radially away from the outer surface by a first distance, and a plurality of second projections extending radially away from the outer surface by a second distance, the second distance being less than the first distance.

Example 6: the lead assembly of example 5, wherein each second projection of the plurality of second projections is separated from another second projection around an outer perimeter of the spline body by one or more first projections of the plurality of first projections.

Example 7: the lead assembly of any of examples 1-6, further comprising: a plurality of slip tubes, each slip tube of the plurality of slip tubes comprising an elongated body defining an inner lumen extending along a longitudinal length of the slip tube, wherein each conductor is at least partially disposed within the inner lumen of a respective slip tube of the plurality of slip tubes, and wherein each slip tube of the plurality of slip tubes is configured to be disposed within a respective conductor channel of the plurality of conductor channels.

Example 8: the lead assembly of any of examples 1-7, wherein each electrical contact of the plurality of electrical contacts comprises: an annular body defining an outer surface and an inner surface defining a contact lumen, wherein the outer surface of each electrical contact is configured to contact tissue of a patient; wherein the spline body is configured to be disposed inside the contact lumen of the annular body of each electrical contact, and wherein when the spline body is disposed within the contact lumen of the annular body, a surface of one or more projections of the plurality of projections is configured to contact the inner surface of the annular body.

Example 9: the lead assembly of any of examples 1-8, wherein the plurality of projections are evenly distributed around the outer perimeter of the spline body.

Example 10: a method of constructing a lead assembly, the method comprising: disposing a plurality of conductors within a plurality of conductor channels in a spline, the spline comprising: a spline body extending along a longitudinal axis, and a plurality of projections disposed around an outer perimeter of the spline body, each projection extending radially away from an outer surface of the spline body, wherein one or more projections of the plurality of projections extend along the longitudinal axis, and wherein each projection of the one or more projections comprises two or more projection segments separated by one or more longitudinal gaps, wherein the plurality of projections define the plurality of conductor channels along at least a portion of the longitudinal length of the spline body; disposing a plurality of electrical contacts over the spline body; electrically coupling each conductor of the plurality of conductors to a respective electrical contact of the plurality of electrical contacts; and disposing an outer lead body radially outwards from at least a portion of the spline body and at least a portion of each conductor of the plurality of conductors.

Example 11: the method of example 10, wherein the spline body comprises a plurality of separate spline segments, each separate spline segment comprising: a respective portion of the spline body; and a respective projection segment of the two or more projection segments of each projection of the one or more projections, wherein each spline segment is separated from a longitudinally adjacent spline segment by a respective longitudinal gap, and wherein disposing the plurality of electrical contacts over the spline body comprises disposed an electrical contact over a respective spline segment of the plurality of separate spline segments.

Example 12: the method of example 11, wherein the plurality of projections comprises: a plurality of first projections extending radially away from the outer surface of the spline body by a first distance; and a plurality of second projections extending radially away from the outer surface by a second distance, the second distance being less than the first distance.

Example 13: the method of example 12, wherein each second projection of the plurality of second projections is separated from another second projection around an outer perimeter of the spline body by one or more first projections of the plurality of first projections.

Example 14: the method of any of examples 10-13, wherein disposing the outer lead body over the spline body comprises: flowing a biocompatible polymer around the spline body via a plurality of mold gates disposed within the one or more longitudinal gaps; and overmolding the biocompatible polymer radially outwards from the at least a portion of the spline body and the at least a portion of each conductor of the plurality of conductors.

Example 15: the method of any of examples 10-14, wherein disposing the plurality of conductors within the plurality of conductor channels in the spline body comprises: disposing each conductor of the plurality of conductors within a respective slip tube of a plurality of slip tubes; and disposing each slip tube of the plurality of slip tubes within a respective conductor channel of the plurality of conductor channels.

Example 16: the method of any of examples 10-15, wherein each electrical contact of the plurality of electrical contacts comprises: an annular body defining an outer surface and an inner surface defining a contact lumen, wherein disposing the plurality of electrical contacts over the spline body comprises: disposing each electrical contact over a respective portion of the spline until a surface of one or more projections of the plurality of projections contacts the inner surface of the annular body, and wherein electrically coupling each electrical conductor of the plurality of conductors to the respective electrical contact of the plurality of electrical contacts comprises: affixing a distal end of each electrical conductor to an inner surface of the annular body of the respective electrical contact.

Example 17: the method of example any of examples 10-16, wherein the plurality of projections define the plurality of conductor channels as one or more helixes extending along a longitudinal length of the spline body and around the outer perimeter of the spline body.

Example 18: a method of constructing a spline, the method comprising: forming a spline body extending along a longitudinal axis; forming a plurality of projections extending from an outer surface of the spline body and around an outer perimeter of the spline body; and ablating portions of one or more projections of the plurality of projections until each of the one or more projections defines two or more projection segments separated by one or more longitudinal gaps, wherein the plurality of projections define a plurality of conductor channels along the outer perimeter of the spline body, wherein each conductor channel of the plurality of conductor channels is configured to retain a respective conductor of a plurality of conductors.

Example 19: the method of example 18, wherein forming the plurality of projections comprises forming the plurality of projections along spline body to define the plurality of conductor channels as one or more helixes extending along a longitudinal length of the spline and around the outer perimeter of the spline.

Example 20: the method of example 18, wherein forming the plurality of projections comprises: forming a plurality of first projections extending radially away from the outer surface of the spline body by a first distance; and forming a plurality of second projections extending radially away from the outer surface of the spline body by a second distance, the second distance being less than the first distance.

What is claimed is:

1. A lead assembly comprising:
a plurality of conductors, each conductor of the plurality of conductors extending from a conductor first end to a conductor second end along a longitudinal axis;
a spline extending from a spline first end to a spline second end, the spline comprising:
a spline body extending along the longitudinal axis, and
a plurality of projections disposed around an outer perimeter of the spline body, the plurality of projections extending radially away from an outer surface of the spline body, wherein one or more projections of the plurality of projections extend along the longitudinal axis, and wherein each projection of the one or more projections comprises two or more projection segments separated by one or more longitudinal gaps,
wherein the plurality of projections define a plurality of conductor channels around the outer perimeter of the spline body, wherein each conductor of the plurality of conductors is disposed within a respective conductor channel of the plurality of conductor channels;
a plurality of electrical contacts electrically coupled to respective conductors of the plurality of conductors at the conductor first end; and
an outer lead body disposed radially outwards from at least a portion of the spline body and at least a portion of each conductor of the plurality of conductors.

2. The lead assembly of claim 1, wherein the plurality of projections are configured to electrically isolate a conductor of the plurality of conductors from a circumferentially adjacent conductor of the plurality of conductors.

3. The lead assembly of claim 1, wherein the plurality of projections define the plurality of conductor channels as one or more helixes extending along a longitudinal length of the spline body and around the outer perimeter of the spline body.

4. The lead assembly of claim 1,
wherein the spline body comprises a plurality of separate spline segments, each separate spline segment comprising:
a respective portion of the spline body; and
a respective projection segment of the two or more projection segments of each projection of the one or more projections,
wherein each spline segment is separated from a longitudinally adjacent spline segment by a respective longitudinal gap, and
wherein each separate spline segment longitudinally overlaps with a respective electrical contact of the plurality of electrical contacts.

5. The lead assembly of claim 1, wherein the plurality of projections comprises:
a plurality of first projections extending radially away from the outer surface by a first distance, and
a plurality of second projections extending radially away from the outer surface by a second distance, the second distance being less than the first distance.

6. The lead assembly of claim 5, wherein each second projection of the plurality of second projections is separated from another second projection around an outer perimeter of the spline body by one or more first projections of the plurality of first projections.

7. The lead assembly of claim 1, further comprising:
a plurality of slip tubes, each slip tube of the plurality of slip tubes comprising an elongated body defining an inner lumen extending along a longitudinal length of the slip tube,
wherein each conductor is at least partially disposed within the inner lumen of a respective slip tube of the plurality of slip tubes, and
wherein each slip tube of the plurality of slip tubes is configured to be disposed within a respective conductor channel of the plurality of conductor channels.

8. The lead assembly of claim 1, wherein each electrical contact of the plurality of electrical contacts comprises:
an annular body defining an outer surface and an inner surface defining a contact lumen,
wherein the outer surface of each electrical contact is configured to contact tissue of a patient;
wherein the spline body is configured to be disposed inside the contact lumen of the annular body of each electrical contact, and
wherein when the spline body is disposed within the contact lumen of the annular body, a surface of one or more projections of the plurality of projections is configured to contact the inner surface of the annular body.

9. The lead assembly of claim 1, wherein the plurality of projections are evenly distributed around the outer perimeter of the spline body.

10. A method of constructing a lead assembly, the method comprising:
disposing a plurality of conductors within a plurality of conductor channels in a spline, the spline comprising:
a spline body extending along a longitudinal axis, and
a plurality of projections disposed around an outer perimeter of the spline body, each projection extending radially away from an outer surface of the spline body, wherein one or more projections of the plurality of projections extend along the longitudinal axis, and wherein each projection of the one or more projections comprises two or more projection segments separated by one or more longitudinal gaps,
wherein the plurality of projections define the plurality of conductor channels along at least a portion of the longitudinal length of the spline body;
disposing a plurality of electrical contacts over the spline body;
electrically coupling each conductor of the plurality of conductors to a respective electrical contact of the plurality of electrical contacts; and
disposing an outer lead body radially outwards from at least a portion of the spline body and at least a portion of each conductor of the plurality of conductors.

11. The method of claim 10,
wherein the spline body comprises a plurality of separate spline segments, each separate spline segment comprising:
a respective portion of the spline body; and
a respective projection segment of the two or more projection segments of each projection of the one or more projections,
wherein each spline segment is separated from a longitudinally adjacent spline segment by a respective longitudinal gap, and
wherein disposing the plurality of electrical contacts over the spline body comprises disposed an electrical contact over a respective spline segment of the plurality of separate spline segments.

12. The method of claim 11, wherein the plurality of projections comprises:
a plurality of first projections extending radially away from the outer surface of the spline body by a first distance; and
a plurality of second projections extending radially away from the outer surface by a second distance, the second distance being less than the first distance.

13. The method of claim 12, wherein each second projection of the plurality of second projections is separated from another second projection around an outer perimeter of the spline body by one or more first projections of the plurality of first projections.

14. The method of claim 10, wherein disposing the outer lead body over the spline body comprises:
flowing a biocompatible polymer around the spline body via a plurality of mold gates disposed within the one or more longitudinal gaps; and
overmolding the biocompatible polymer radially outwards from the at least a portion of the spline body and the at least a portion of each conductor of the plurality of conductors.

15. The method of claim 10, wherein disposing the plurality of conductors within the plurality of conductor channels in the spline body comprises:
disposing each conductor of the plurality of conductors within a respective slip tube of a plurality of slip tubes; and
disposing each slip tube of the plurality of slip tubes within a respective conductor channel of the plurality of conductor channels.

16. The method of claim 10,
wherein each electrical contact of the plurality of electrical contacts comprises:
an annular body defining an outer surface and an inner surface defining a contact lumen,
wherein disposing the plurality of electrical contacts over the spline body comprises:
disposing each electrical contact over a respective portion of the spline until a surface of one or more projections of the plurality of projections contacts the inner surface of the annular body, and
wherein electrically coupling each electrical conductor of the plurality of conductors to the respective electrical contact of the plurality of electrical contacts comprises:
affixing a distal end of each electrical conductor to an inner surface of the annular body of the respective electrical contact.

17. The method of claim 10, wherein the plurality of projections define the plurality of conductor channels as one or more helixes extending along a longitudinal length of the spline body and around the outer perimeter of the spline body.

18. A method of constructing a spline, the method comprising:
forming a spline body extending along a longitudinal axis;
forming a plurality of projections extending from an outer surface of the spline body and around an outer perimeter of the spline body; and
ablating portions of one or more projections of the plurality of projections until each of the one or more projections defines two or more projection segments separated by one or more longitudinal gaps,
wherein the plurality of projections define a plurality of conductor channels along the outer perimeter of the spline body, wherein each conductor channel of the plurality of conductor channels is configured to retain a respective conductor of a plurality of conductors.

19. The method of claim 18, wherein forming the plurality of projections comprises forming the plurality of projections along spline body to define the plurality of conductor channels as one or more helixes extending along a longitudinal length of the spline and around the outer perimeter of the spline.

20. The method of claim 18, wherein forming the plurality of projections comprises:
forming a plurality of first projections extending radially away from the outer surface of the spline body by a first distance; and
forming a plurality of second projections extending radially away from the outer surface of the spline body by a second distance, the second distance being less than the first distance.
